(19) United States
(12) Patent Application Publication
(10) Pub. No.: US 2022/0072107 A1
(43) Pub. Date: Mar. 10, 2022

(54) NUTRITIONAL COMPOSITION FOR INDUCING A FEELING OF SATIETY, A BETTER SLEEP AND/OR LIMITING NOCTURNAL AWAKING IN INFANTS OR YOUNG CHILDREN

(71) Applicant: SOCIETE DES PRODUITS NESTLE S.A., Vevey (CH)

(72) Inventors: Lionel Jean Rene Bovetto, Lucens (CH); Jonathan O'Regan, Killarney (IE); Sophie Pecquet, La Conversion (CH)

(21) Appl. No.: 17/416,584

(22) PCT Filed: Dec. 6, 2019
(86) PCT No.: PCT/EP2019/083932

§ 371 Date: Jun. 21, 2021

(30) Foreign Application Priority Data
Dec. 21, 2018 (EP) 18215528.3

(51) Int. Cl. A61K 38/40; A61K 38/17; A61K 31/201; A61K 31/202; A61K 33/26; A61K 33/30; A61K 33/06; A61K 31/59; A61K 33/00; A61K 31/714; A61K 38/38; A61K 38/01; A23L 33/00; A23L 33/19; A23L 33/12; A23L 33/125; A23L 33/18; A23L 33/16; A23L 33/155

(52) U.S. Cl. CPC A61K 38/40; A23V 2002/00; A61K 31/201; A61K 31/202; A61K 33/26; A61K 33/30; A61K 33/06; A61K 31/59; A61K 33/00; A61K 31/714; A61K 38/38; A61K 38/018; A23L 33/40; A23L 33/19; A23L 33/12; A23L 33/125; A23L 33/18; A23L 33/16; A23L 33/155; A61K 38/1722

(57) ABSTRACT

The invention relates to a nutritional composition for infants and young children comprising proteins, carbohydrates and lipids, wherein said proteins comprise ionic complexes of lactoferrin with acid milk proteins, said complexes having a negative charge at the pH of the infant formula. The nutritional composition is used for providing a satiety feeling and/or a better sleep and/or limiting nocturnal awaking in infants or young children.

### TECHNICAL FIELD

The present invention relates to a nutritional composition, for infants or young children, specifically adapted for night feeding, containing a formula able to mimic the effect of the night human milk.

The nutritional composition of the invention comprises ionic complexes of lactoferrin with acid milk proteins, said complexes having a negative charge at the pH of the infant formula.

The composition combines a nutritional content and a food structure organized in functional blocks close to human milk, which provides satiety and gut comfort in order to improve circadian cycle, for a better sleep, limiting nocturnal awaking.

### BACKGROUND OF THE INVENTION

The invention concerns nutritional compositions and uses thereof in nutritional compositions for infants and young children for inducing a feeling of satiety, a better sleep and/or limiting nocturnal awaking. The nutritional compositions comprise proteins, carbohydrates and lipids. The nutritional composition of the invention is a synthetic composition, i.e. a man-made nutritional composition.

Breast feeding is considered as the ideal source of nutrition and is the preferred choice for feeding infants up to at least 6 months of age. Consequently, human milk (HM) has long been considered as the model for the design of infant formulas (IF). Even many improvements in the nutrient composition of IF have been made during the last decades, there are still important differences in composition as well as in functional benefits conveyed by HM.

The most common sleep disturbances in infants and children are those related to wakefulness (i.e. either difficulties in settling at bedtime or failure to sleep through the night without interruptions). It has been estimated that these disturbances affect 15 to 35% of infants aged less than 24 months (France et al, “Infant Sleep Disturbance: Description of a problem behaviour process”, Sleep Medicine Reviews, Vol 3, No 4, pp 265-280, 1999).

Sleep-wake regulation and sleep states evolve rapidly during the first year of life with continued maturation across childhood. At around 10-12 weeks of age, the circadian rhythm begins to emerge, and infant sleep becomes increasingly nocturnal. Sleep-wake patterns are also influenced by the interaction of biological processes and environmental, behavioral and social factors. Age-specific data from sleep measures in relation to the quantity of sleep over a 24 h period, the number of episodes of waking during nocturnal sleep, the time taken to fall asleep (sleep latency), the longest sleep over a 24 h period and the number of daytime naps, from birth to the age of 12, is reported in B. C. Galland et al., Normal sleep patterns in infants and children: A systematic review of observational studies, Sleep Medicine Reviews, 2012, 213-222. Sleep quality (and thus improvement of sleep quality) can be measured by any conventionally known method, for example as per the above parameters as well as by the measure of REM/NREM periods (rapid eye movement periods/non rapid eye movement periods).

It has also been reported that sleep schedules shift increasingly with development and implications of sleep timing for obesity appear to begin at least during early childhood (C A Magee et al., The longitudinal relationship between sleep duration and body mass index in children: a growth mixture modeling approach. J Dev Behav Pediatr. 2013, 34:165-173).

There is thus a need for nutritional compositions that helps securing good sleep quality and/or normal sleep time for infants and young children. More particularly there is a need for nutritional compositions delivering such benefits while also securing the most adequate energy balance and nutrients intake.

Lactoferrin is an iron-binding glycoprotein, which is a major component of human breast milk. It is considered to have a range of biological functions in infants, including roles in gut maturation, immune development, prevention of infections and iron absorption. Lactoferrin is present at very high levels in human colostrum (up to 10 g/l has been reported), with levels in mature human milk decreasing significantly as the infant ages (2-3 g/l at 1 month, 1 g/l at 6 months). Lactoferrin has been of interest for use in infant formula for some time but the high cost has generally prevented its use.

The use of lactoferrin in infant formula as a dietary ingredient for promoting the growth of the gastrointestinal tract is disclosed EP 0 295 009.

WO2011/051482 relates to nutritional composition for infants and/or children comprising lactoferrin and probiotics for providing health benefits.

It has been recently shown that milk composition, and in particular maternal-origin lactoferrin which is produced in mammary epithelial cells and secreted into milk, naturally varies as a function of infant's health and needs (A. A Breakey et al., Evolution, Medicine and Public Health, 2015, volume 2015, 21-31).

The biological activity of lactoferrin is sensitive to its structure. It can be modulated by interactions with various components of body fluids, in particular human milk.

The addition of pure lactoferrin in a nutritional formulation is often difficult because lactoferrin, is difficult to disperse in the presence of acid milk proteins, such as casein or whey protein, because lactoferrin has an isoelectric point of about pH 9, while casein or whey proteins have an isoelectric point of approximately pH 5.

Therefore, addition of lactoferrin may result into aggregates formation in the mixing container, leading to a non-homogeneous dispersion. It is thus wanted to obtain a homogeneous formulation as regards, in particular, dispersion of the protein components, while providing the adequate amount of lactoferrin in the nutritional composition.

Alpha-Lactalbumin (ALAC) is the major protein in Human breast milk (20-25% of total proteins), associated with the whey fraction. It is also present in cow milk, though at a much lower level (2-5% of total proteins). ALAC is a small (MW≈14 KDa) acidic protein. ALAC offers interesting health benefits, such as anti-microbial/infection, immunomodulatory, anti-hypertensive, anti-tumour, anti-oxidant, promotion of cognitive function. Tryptophan bioavailability from ALAC was reported in infants (W. Heine et al., alpha-Lactalbumin-enriched low-protein infant formulas: a comparison to breast milk feeding, Acta Paediatr. 1996, 85, 1024-8).

Studies examining the effect of tryptophan have suggested that its intake does modulate both the quality and quantity/duration of sleep.

Tryptophan is an essential amino acid whereby its influence on sleep has been related to its role in the synthesis of brain neurotransmitter serotonin). To exert this effect, tryptophan has to cross the blood-brain barrier where it is utilized to synthesize serotonin, which, in turn, gets converted into melatonin. Serotonin is the neurotransmitter that affects mood and appetite, as well as other processes in the body, while melatonin is the hormone vital to facilitating sleep (N. Schneider et al., Diet and nutrients in the modulation of infant sleep: A review of the literature, Nutritional Neuroscience, 2018, 21, 151-161).

The beneficial effect of ALAC in human nutrition and its effect on regulation of sleep/wakes cycles has been reported in D. K. Layman et al., Applications for α-lactalbumin in human nutrition, Nutr Rev. 2018, 76, 444-460, which mentions that that dietary tryptophan influences the synthesis of both the neurotransmitter serotonin in the brain and the hormone melatonin in the intestines, which are involved in regulating sleep, and that tryptophan levels in breast milk are maximal during the night. The authors conclude that the effect of alpha-lactalbumin—enriched formulas on infant sleep patterns has not been examined.

Research is ongoing on the relationship between micronutrient status and sleep patterns (J. Xiaopeng et al., Public Health Nutr, 2017, 20(4), 687-701).

It has furthermore been suggested that, in breast milk, certain nutrients follow a circadian rhythm, suggesting a role in supporting the infant's sleep/wake cycle development and maturation (N. Schneider et al., Nutritional Neuroscience, 2018, 21, 151-161). In particular, the levels of inducing compounds such as tryptophan, nucleotides, hormones, and neurotransmitters fluctuate over a 24-hour period and maximum levels of tryptophan and melatonin are reached during the night.

Therefore, there is a need for further developing infant and young children nutritional compositions specifically adapted for night feeding, which formula is able to mimic the effect of night human milk, which is different from day human milk.

There is a need to provide infant and young children nutritional compositions specifically adapted for night feeding, whereby chronobiological requirements are taken into consideration by providing nutrients and micronutrients at the time when they promote better sleep and limit awaking.

It is therefore an object of the invention to provide a nutritional composition, adapted for night feeding, which can be used as a daily diet solution adapted to the status of the infant or young child (circadian cycle, gut comfort, etc.) and provides the desired feeling of satiety and effect on sleep.

In addition, it is an object of the invention to provide a nutritional composition according which ensures a balanced nutritional intake, and avoid overfeeding due to extra regular formula bottle at night.

There is generally a need to deliver the above mentioned benefits without impacting any other health parameters such as growth, development of the immune system, cognitive development or gastrointestinal functions.

There is a need to deliver the above mentioned benefits while enhancing the other health parameters, for example enhancing indirectly the cognitive development (via high sleep quality) and enhancing the intestinal/digestive functions (good sleep quality being also linked to easier digestion).

There is a need to deliver the above mentioned benefits in the context of a nutritional composition for infants, especially young infants, as part of a nutritional intervention and not as part of a pharmaceutical intervention (separate intake of medicaments).

### SUMMARY OF THE INVENTION

It has now been found that a nutritional composition in which lactoferrin is organized in the form of ionic complexes with milk proteins could be beneficial for providing the desired feeling of satiety in infants and young children, and to modulate the quality and quantity of sleep, in particular by providing adequate levels of micronutrients, such as tryptophan, thus avoiding or limiting night awaking.

Advantageously, said ionic complexes of lactoferrin and ionic complexes with milk proteins possess a structure which is close to that naturally occurring in human milk.

Without wishing to be bound by theory, it is hypothesized that said ionic complexes of lactoferrin with milk proteins may play a role in providing slow digestion of proteins, in particular whey proteins such as alpha-lactalbumin and beta-lactoglobulin, as well as casein and of lipids, and adequate release of micronutrients, such as minerals and vitamins, thereby providing the desired feeling of satiety and effect on sleep.

In particular, it has been found that minerals and vitamins could be adapted to favour sleep by enhancing the micronutrients which favour sleep, such as Fe, Zn, Mg and vitamin D, and reducing the micronutrients which negatively impact sleep, such as K or vitamin B12, while maintaining an optimal nutritional balance.

In a first aspect of the invention, there is provided a nutritional composition for infants and young children comprising proteins, carbohydrates and lipids, wherein said proteins comprise ionic complexes of lactoferrin with acid milk proteins, said complexes having a negative charge at the pH of the infant formula, namely pH 7.0±0.5.

In particular, said acid milk protein is selected from the group consisting of alpha-lactalbumin (ALAC), beta-lactoglobulin (BLG), whey protein isolate (WPI), hydrolysed WPI, casein and combinations thereof.

In a second aspect, the nutritional composition of the invention is used for providing a satiety feeling and/or a better sleep and/or limiting nocturnal awaking in infants or young children. Advantageously, the nutritional composition provides slow digestion of whey proteins, and adequate release of micronutrients.

According to one embodiment, the nutritional composition of the invention is used in infants or young children having sleep disorders with respect to sleep quality or sleep time.

According to one embodiment, the nutritional composition according to the invention can be, for example, an infant formula, a starter infant formula or a follow-on or follow-up formula.

The invention is particularly suitable for induce a more mature sleep pattern in infants and thus improve their sleep quality and reduce the episodes of wakefulness. In one embodiment, the invention relates to reducing sleep disturbance and/or improving sleep patterns in infants or young children.

In one embodiment the improvement of sleep quality or pattern is characterized, comprised or is limited to the reduction of the number of episodes of wake states and/or the reduction of sleep fragmentation.

In one embodiment the improvement of sleep quality is characterized by longer nights without being unwillingly awake and by a more peaceful sleep.

In one embodiment the improvement of sleep quality is characterized by better ability to fall asleep.

### BRIEF DESCRIPTION OF THE FIGURES

FIG. 1 shows the variation of the Zeta-potential of complexes of lactoferrin (LF) and alpha-lactalbumin (ALAC) for variable concentrations of ALAC at pH 6.0. On the left side, the Zeta potential of LF alone is shown by the black triangle and that of ALAC alone is shown by the black circle.

FIG. 2 shows the variation of the Zeta-potential of complexes of LF and ALAC as a function of pH.

FIG. 3 shows the variation of the Zeta-potential of complexes of LF and ALAC as a function of pH after freeze and thaw.

FIG. 4 shows the variation of the Zeta-potential of complexes of LF (0.1%) and beta-lactoglobulin (Blg) for variable concentrations of Blg at pH 7.0.

FIG. 5 shows the variation of the Zeta-potential of complexes of LF (1%) and beta-lactoglobulin (Blg) for variable concentrations of Blg at pH 7.0.

FIG. 6 shows the variation of the Zeta-potential of complexes of LF (0.1%) and tryptic whey protein hydrolyzate (Lactry) for variable concentrations of Lactry at pH 7.0.

FIG. 7 shows the variation of the Zeta-potential of complexes of LF (0.1%) and tryptic whey protein hydrolyzate (Lactry) for variable concentrations of Lactry at pH 7.0.

FIG. 8 shows the variation of the Zeta-potential of complexes of LF (2%) and beta-lactoglobulin (Blg) for variable concentrations of Blg at pH 7.0.

FIG. 9 shows the variation of the Zeta-potential of complexes of LF (10%) and beta-lactoglobulin (Blg) for variable concentrations of Blg at pH 7.0.

### DETAILED DESCRIPTION

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to.

All percentages disclosed herein are on a w/w basis, unless stated otherwise.

Unless defined otherwise, all technical and scientific terms used herein have the same meaning as commonly understood by one of ordinary skill in the art.

The term “infant” will, in the context of the present invention, mean a child under the age of 12 months.

The term “young child” refers to a child in the age from 12 months to 3 years.

In the context of the present invention, the infant may be any term infant or preterm infant. In an embodiment of the invention, the infant is selected from the group of preterm infants and term infants.

The term “infant formula” (or “IF”) as used in the context of the present invention refers to a nutritional composition intended for infants during the first months of life and as defined in Codex Alimentarius, (Codex STAN 72-1981) and Infant Specialties (incl. Food for Special Medical purposes) as defined in Codex Alimentarius, (Codex STAN 72-1981).

The term “follow-on formula” or “follow-up formula” refers to formulas designed to be used from the age of 6 months onwards, generally up to 12 months of age. It constitutes the principal liquid element in the progressively diversified diet of infants.

The term “growing-up milk” is given to formulas designed to be used from the age of one year onwards, generally until three years of age. It is generally a milk-based beverage adapted for the specific needs of young children.

The term “composition for use in formulas for infants or young children” refers in the context of the present invention to either a formula as such, i.e., an infant formula (IF), which comprises all nutrients necessary in order to meet the standards of being an infant formula as defined in the Codex Alimentarius. Further, the “composition for use in formulas for infants or young children” may be a composition comprising nutrients, which, together with other nutrients, can be mixed to prepare a formula, i.e. such “composition for use in infant formulation” can be added to a mixture, which is intended to be used as an infant formula.

The -terms “ionic complexes of lactoferrin with milk protein(s)” and “electrostatic complexes of lactoferrin with milk protein(s)” will be used equally throughout the description.

Proteins:

The nutritional composition of the invention may contain a protein source in an amount of less than or equal to 2.5 g/100 kcal, preferably 1.68 to 2.3 g/100 kcal, most preferably 1.8 to 2.2 g/100 kcal.

The type of protein is not believed to be critical to the present invention, provided that the minimum requirements for essential amino acid content are met and satisfactory growth is ensured.

In some advantageous embodiments, the protein source in the nutritional composition is whey predominant (i.e. more than 50% of proteins are coming from whey proteins, such as 60% or 70%). In another embodiment, the protein content is between 35 and 70% whey proteins.

In particular, whey proteins can be used in the form of “whey protein micelles” as disclosed in EP183492, in liquid concentrate or powder form.

Protein sources based on whey, casein and mixtures thereof may be used as well as protein sources based on soy.

In one embodiment of the invention, the nutritional composition comprises protein that is a mixture of whey protein and casein wherein the ratio of whey protein to casein is between 50:50 and 80:20. For example, the ratio of whey protein to casein may be 70:30 for a starter formula or 50:50 for a follow-up formula

As far as whey proteins are concerned, the protein source may be based on acid whey or sweet whey or mixtures thereof, and may include alpha-lactalbumin and/or beta-lactoglobulin in whatever proportions are desired.

In particular, whey protein having an alpha-lactalbumin content of at least 10% by weight, preferably at least 14%, more preferably at least 20%, at least 25%, at least 30%, or at least 40%, or at least 45% or at least 50% can be used. By “whey protein is enriched with alpha-lactalbumin (ALAC)» it is to be understood in the context of the present invention that the content of the ALAC in the whey protein is higher than what is naturally present in the raw material from which the whey protein is separated/extracted.

For example, if ALAC is naturally present at around 1.2-1.5 g/L in bovine milk and accounts for about 18-20% of the total whey proteins, any proportion higher than that represents an enrichment.

For example, whey protein enriched with alpha-lactalbumin (ALAC), as disclosed in WO2018202636 or WO2003055322, can be used in this respect.

In one embodiment of the invention, the source of whey proteins is enriched with complexes of lactoferrin with acid milk proteins such as alpha-lactalbumin (ALAC) and/or beta-lactoglobulin (BLG), as indicated below.

In one embodiment of the invention, the source of proteins is enriched with complexes of lactoferrin with casein, as indicated below.

Modulation of the protein composition, particularly by increasing the lactalbumin content and/or providing complexes of lactoferrin with acid milk proteins makes it possible to mimic the composition of human milk, and to provide a beneficial effect on promoting a slow release of tryptophan content, of which the activity on sleep is well documented.

Lactoferrin can be human colostrum lactoferrin, human milk lactoferrin or bovine milk lactoferrin or lactoferrin of other source. A preferred source of lactoferrin is bovine milk lactoferrin that has been shown to provide the expected benefits when incorporated into the composition of the invention.

The lactoferrin can be isolated from animal milk or can be a recombinant form of lactoferrin (such as recombinant human lactoferrin or recombinant bovine lactoferrin). The lactoferrin considered in the present invention can be pure isolated lactoferrin (or having a high degree of purity). In one embodiment the lactoferrin is comprised in a lactoferrin-rich fraction and is accompanied by other nutrients. The lactoferrin can be in a lactoferrin-rich fraction of bovine milk (by “rich” is meant that the content in lactoferrin is high than in the native ingredient). Lactoferrin is commercially available and can be sourced, for example, from DMV International (Netherlands), Murray Goulburn (Australia), Tatua (New Zealand), Fonterra (New Zealand), Milei/Morinaga (Germany/Japan).

Due to its high isoelectric point, lactoferrin has a positive charge in the physiological pH conditions of milk, which enables non-specific electrostatic binding to, inter alia, other milk proteins during extraction of lactoferrin from milk, product processing or digestion.

Ionic complexes of lactoferrin and acid milk proteins (i.e proteins having an isoelectric point lower than pH 7.0) can be prepared, for example, as disclosed in WO 2012/045801. However, the ionic complexes of lactoferrin and acid milk proteins which can be used according to the invention can be prepared without being bound by the physicochemical requirements (ionic strength, temperature, etc.) defined for obtaining the coacervates of WO 2012/045801.

For example, said ionic lactoferrin complexes can be prepared as follows:
preparing a protein solution by dispersing lactoferrin at a concentration of 1 to 20% in water
preparing a protein solutions dispersing an acid milk protein or a combination of acid milk proteins at a concentration of 20 to 50%,
adjusting the pH of each solution at the same pH, preferably between 6.0 and 7.0,
mixing the two protein solutions according to pre-determined weight ratios.

The preparation can be carried-out at a temperature of 4° C. to 65° C., preferably at ambient temperature, such as 15° C. to 25° C.

In one embodiment, the weight ratio of lactoferrin to acid milk protein ranges from 1:0.25 to 1:3, in particular 1:1, 1:1.5 or 1:2.

Advantageously, lactoferrin is added to the nutritional composition in the form of complexes with acid milk proteins, wherein said complexes have a negative charge at the pH of the infant formula, i.e. pH 7.0±0.5.

Preferably, the Zeta-potential of said complexes at pH 7.0 is from −3 mV to −20 mV, preferably from −10 mv to −15 mV measured for a total protein content of 0.1%.

The negative charge can be modulated as a function of the pH.

Advantageously, it has been shown that the ionic complexes of lactoferrin and acid milk protein(s) according to the invention complexes were stable (maintenance of Zeta-potential) after undergoing freezing and thawing, a physical process which is known to strongly affect protein structure.

Said acid milk protein may be selected, for example, from the group consisting of alpha-lactalbumin (ALAC), beta-lactoglobulin (BLG), whey protein isolate (WPI), Whey protein concentrate (WPC), hydrolyzed whey protein, casein and combinations thereof, which are commercially available.

As stated above, “whey protein” and products deriving therefrom may be provided as a preparation in which, for example, alpha-lactalbumin and/or beta-lactoglobulin are present in whatever proportions are desired.

All types of caseins can be used for the purpose of the present invention. β-Casein, κ-casein and α-s1 casein are however preferred, more preferably β-Casein, for addition in products for infants and young children, such as infant formula or follow-on formula, in so far as they are naturally present in human breast milk. In a preferred aspect, casein is in the form of micellar casein.

In one embodiment of the invention, lactoferrin is added to the nutritional composition in the form of complexes with acid milk protein(s) where:
the weight ratio of lactoferrin to ALAC ranges from 1:0.25 to 1:3, e.g 1:1, 1:1.5 or 1:2; and/or
the weight ratio of lactoferrin to BLG ranges from 1:0.25 to 1:3, e.g 1:1, 1:1.5 or 1:2; and/or
the weight ratio of lactoferrin to casein ranges from 1:0.25 to 1:3, e.g 1:1, 1:1.5 or 1:2.

In one embodiment, lactoferrin in the form of ionic complexes with acid milk protein(s) may represent 1% to 20%, preferably 2 to 10% by weight of the whey protein.

In one embodiment, lactoferrin in the form of ionic complexes with casein may represent 1% to 20%, preferably 2 to 10% by weight of total protein content.

The amount of lactoferrin in the present composition is preferably between 2 g and 0.12 g per liter of reconstituted nutritional composition (or per liter of ready-to-feed/ready-to—drink liquid composition). In the powder form of the composition, the amount of lactoferrin can be, for example, between 1.6 to 0.4 g of dry composition (w/w).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

It may be desirable to supply partially hydrolyzed proteins (degree of hydrolysis between 2 and 20%), for example for infants believed to be at risk of developing cows' milk allergy. If hydrolyzed proteins are used, the hydrolysis process may be carried out as desired and as is known in the art. For example, a whey protein hydrolysate may be prepared by enzymatically hydrolyzing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

Lipids:

The nutritional composition may contain lipids in an amount of lower than or equal to 6 g/100 kcal, for example 4.0 to 6 g/100 kcal.

This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to 15:1, for example about 8:1 to 12:1.

Preferably, the nutritional composition contains linoleic acid and/or linolenic acid and/or arachidonic acid (ARA) and/or docosahexaenoic acid (DHA).

Carbohydrates

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

Lactose may represent at least 90%, preferably at least 98% of the carbohydrate present in the composition.

Minerals and Vitamins

The nutritional composition may also comprise all vitamins and minerals understood to be essential in the daily diet in nutritionally significant amounts. Minimum requirements have been established for certain vitamins and minerals. Examples of minerals, vitamins and other nutrients present in the nutritional composition include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorus, iodine, iron, magnesium, copper, zinc, manganese, chloride, potassium, sodium, selenium, chromium, molybdenum, taurine and L-carnitine. The minerals are usually added in the salt form.

In one embodiment of the invention, minerals and vitamins can be adapted to favour sleep by enhancing the micronutrients which favour sleep, for example, iron (Fe), zinc (Zn), magnesium (Mg) and vitamin D.

In another embodiment of the invention, minerals and vitamins can be adapted to reduce the micronutrients which negatively impact sleep, for example as potassium (K) or vitamin B12.

Accordingly, the amounts of Fe, Zn, Mg and vitamin D, taken individually or in combination, may range as follows:
Fe: 1 to 2, preferably 1.2 to 1.8 mg/100 kcal,
Zn: 0.7 to 1.5, preferably 1 to 1.5 mg/100 kcal,
Mg: 5.5 to 16.5, preferably 10 to 15 mg/100 kcal,
Vitamin D: 1 to 2.6, preferably 1.3 to 2.5 μg/100 kcal.

and the amounts of K or vitamin B12, taken individually or in combination, may range as follows
K: 70 to 170, preferably 70 to 100 mg/100 kcal,
vitamin B12: 0.25 to 1.5, preferably 0.25 to 0.8 μg/100 kcal.

Other minerals and vitamins can be adapted according to the age, needs and/or sleep status of the infant or young child.

Other Nutrients:

If necessary, the composition of the invention may contain emulsifiers and stabilizers such as soy, lecithin, citric acid esters of mono- and di-glycerides, and the like.

The composition may also contain other substances which may have a beneficial effect such as nucleotides, nucleosides, gangliosides, polyamines and the like.

Energy Content

Caloric density for infants is regulated for infant formula between 60-70 kcal/100 ml (Codex STAN 72-1981).

Advantageously, the nutritional composition of the invention delivers the described benefits while delivering an appropriate energy content. Indeed, it may be beneficial to induce better sleep of the infant without increasing the total energy consumed, especially before sleep time, as it may impact negatively the overall growth and health of the infant.

The invention thus relates to delivering the sleep benefits without impacting the growth of the infant nor overloading the digestive system with nutrients in a quantity or quality that would be long or difficult to digest.

In one embodiment, the energy density of the composition of the invention is less than 65 kcal/100 ml or preferably less than 62 kcal/100 ml, or between 60 and 65 kcal/100 ml, or preferably between 60 and 62 kcal/100 ml.

In one embodiment, the nutritional composition of the invention delivers the described benefits while delivering an appropriate energy content. Indeed, it may be beneficial to induce better sleep of the infant without increasing the total energy consumed, especially before sleep time, as it may impact negatively the overall growth and health of the infant. The invention thus relates to delivering the sleep benefits without impacting the growth of the infant and without overloading the digestive system with nutrients in a quantity or quality that would be long or difficult to digest.

In one embodiment, the energy density of the composition of the invention is less than 65 kcal/100 ml or preferably less than 62 kcal/100 ml, or between 60 and 65 kcal/100 ml, or preferably between 60 and 62 kcal/100 ml.

Forms of the Nutritional Compositions for Infants and Young Children:

According to one embodiment of the invention, the nutritional composition is an infant formula.

The infant formula according to the present invention may be a starter formula for infants from the age of birth to 4 to 6 months and which provide complete nutrition for this age group (both for term and preterm infants). Further, the infant formula may be a follow-on formula for infants between the ages of four to six months and twelve months which are fed to the infants in combination with increasing amounts of the foods, such as infant cereals and purée de fruits, vegetables and other foodstuffs as the process of weaning progresses.

According to one embodiment of the invention, the nutritional composition is a growing up milk.

The nutritional composition of the present invention can be in solid (e.g. powder), liquid or gelatinous form. In particular, the nutritional compositions of the invention can be in the form of dehydrated powders which are prepared for consumption by reconstitution with water or milk.

The nutritional compositions of the invention can be in a fluid (liquid) form. These can be sold ready to consume (without further dilution).

The daily dose unit of nutritional composition according to the invention may be disposable capsules equipped with opening means contained within the capsule to permit draining of the reconstituted formula directly from the capsule into a receiving vessel such as a bottle. Such a method of using capsules for dispensing an infant or young child nutritional composition is described in WO2006/077259. The different nutritional compositions may be packed into individual capsules and presented to the consumer in multipacks containing a sufficient number of capsules to meet the requirements of infants and young children one week for example. Suitable capsule constructions are disclosed in WO2003/059778.

Preparation of Nutritional Compositions:

The nutritional compositions according to the present invention may be prepared by any known or otherwise suitable manner. For example, an infant formula may be proposed by blending together a source of protein with a carbohydrate source and a lipid source in appropriate proportions. If used, emulsifiers may be included at this stage. Vitamins and minerals may be added at this stage, but may also be added later to avoid thermal degradation. Water, preferably water which has been subjected to reverse osmosis or deionized water, may then be added and mixed in to form a liquid mixture. The temperature of mixing is preferably room temperature, but may also be higher. The liquid mixture may then be thermally treated to reduce bacterial loads. The mixture may then be homogenized.

If it is desired to produce a powdered composition, the homogenized mixture is dried in a suitable drying apparatus, such as a spray drier or freeze drier and converted into powder.

Processes used in the manufacture of formulae for infants and young children are based on the concept that the products must be nutritionally adequate and microbiologically safe to consume. Thus, steps that eliminate or restrict microbiological growth are central to production processes. The processing technology for each specific formula is proprietary to the manufacturer but, in general, it involves the preservation of an oil-in-water (o/w) emulsion by dehydration in the case of powder products or, sterilization in the case of ready-to-feed or concentrated liquid products. Powdered infant formula may be produced using various processes, such as dry blending dehydrated ingredients to constitute a uniform formula or hydrating and wet-mixing a mixture of macro-ingredients, such as fat, protein and carbohydrate ingredients and then evaporating and spray drying the resultant mixture. A combination of the two processes described above may be used where a base powder is first produced by wet-mixing and spray drying all or some of the macro-ingredients and then dry blending the remaining ingredients, including carbohydrate, minerals and vitamins and other micronutrients, to create a final formula.

Liquid formulae are available in a ready-to-feed format or as a concentrated liquid, which requires dilution, normally 1:1, with water. The manufacturing processes used for these products are similar to those used in the manufacture of recombined milk.

If it is desired to produce a liquid infant formula, the homogenized mixture is filled into suitable containers, preferably aseptically. However, the liquid composition may also be retorted in the container, suitable apparatus for carrying out the filling and retorting of this nature is commercially available.

The invention is further described with reference to the following examples. It will be appreciated that the invention as claimed is not intended to be limited in any way by these examples.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.”

Examples 1-4 relate to zeta potential measurements of complexes of Lactoferrin and alpha-lactalbumin (ALAC), beta-lactoglobulin (Blg) or a whey protein hydrolysate (Lactry). Examples 5 and 6 relate to nutritional compositions according to the invention.

Zeta Potential Measurements

Materials

Alpha-lactalbumin (ALAC) (Lot 001-8-415-6-922) was purchased from Davisco Foods International, Inc. (Le Sueur, Minn., USA). Protein content was 89.6% (Kjeldahl analysis: N×6.38).

Beta-Lactoglobulin (Blg) (Lot JE 003-6-922) was purchased from Davisco Foods International, Inc. (Le Sueur, Minn., USA). Protein content was 90.3% and the purity 97% (given by the supplier).

Lactoferrin (LF) (Lot 10376514) was purchased from DMV (the Netherlands). Protein content was 96.5% and the purity 88.2% (given by the supplier)

Lactry (tryptic whey protein hydrolysate) was purchased from ARLA Foods (DK) (Lot 26996513) Protein content was determined by Kjeldahl analysis (Nt×6.38).

Methods:

Protein solutions (ALAC, Blg, LF, or Lactry,) were prepared by dispersion of wt % (purity based) sample powder in Millipore water (18.2 MΩ·cm) and the pH was adjusted at pH 7.0 with NaOH 1M or HCl 1M.

The dispersion was filtered at 0.22 μm (Millipore Stericup&Steritop) and incubated overnight at 4° C. The final preparation at the desired concentration was made at that time.

Depending on desired concentration ranges (1, 2, 10 or 20%) and wt ratios, stock solutions were prepared for LF, ALAC and Blg and pH was adjusted at pH 7.0 with NaOH 1M or HCl 1M.

The final prepared quantity was 250 ml.

The Zeta potential was determined by light scattering upon application of an alternating electrical field into the measuring cell at a protein concentration of 0.1 wt % after filtration on 0.22 μm filters. The dispersions at the selected pH's were placed in an electrophoretic mobility cell and analyzed at a scattering angle of 173° using the Nanosizer ZS (Malvern Instruments, UK) equipped with a 633 nm laser. The effective electrical field, E, applied to the measurement cell was between 50 and 150 V depending on the conductivity of the samples. The overall mobility of the particles was determined and the corresponding Zeta potential (V) was then calculated using the Smoluchowski equation.

### EXAMPLE 1

### Complexes of Lactoferrin (LF) and Alpha-Lactalbumin (ALAC)

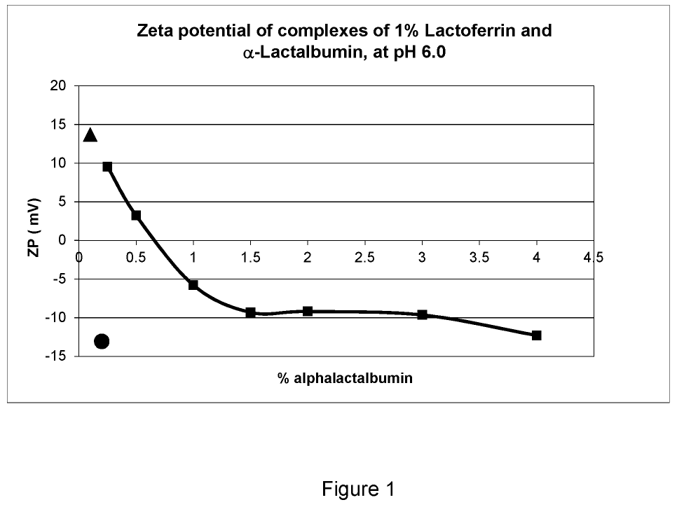

The variation of the Zeta-potential of complexes of lactoferrin (LF) and alpha-lactalbumin (ALAC) for variable concentrations of ALAC at pH 6.0 is shown on FIG. 1. The results show that LF and ALAC can form electrostatic complexes. By adding ALAC (1 wt % to 4 wt %) to LF 1 wt %, the initial Zeta potential (+13 mV) is neutralized, indicating the formation of electrostatic complexes, until a charge saturation is observed when the Zeta potential reaches approximately −10 mV.

### Example 2

### Complexes of Lactoferrin (LF) and Alpha-Lactalbumin (ALAC) Prepared at a 20% Protein Content

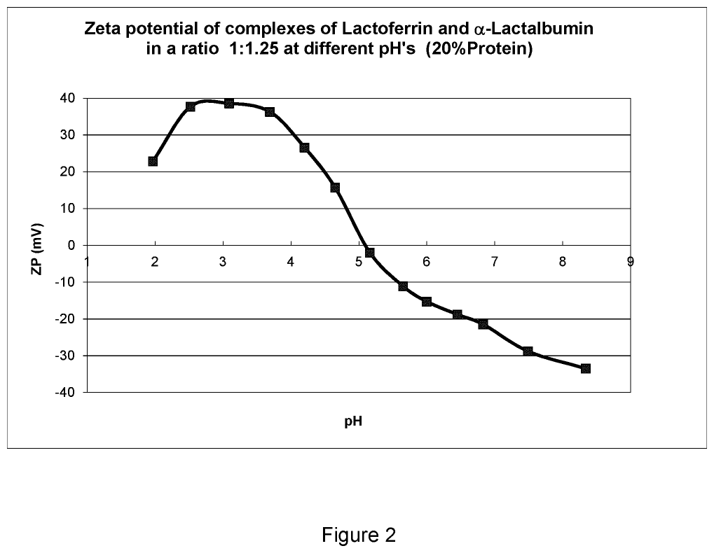

1) Complexes of LF and ALAC prepared at a 20% protein content in a 1:1.25 ratio The variation of the Zeta-potential of complexes of LF and ALAC prepared at a 20% protein content in a 1:1.25 ratio at different pH's is shown on FIG. 2.

For Zeta-potential measurement, the sample was diluted to a 0.1% protein content. The results show that the complexes are negatively charged in the range of pH 5.5 to 8.0.
2) Complexes of LF and ALAC prepared at a 20% protein content in a 1:1.25 ratio after freezing and thawing

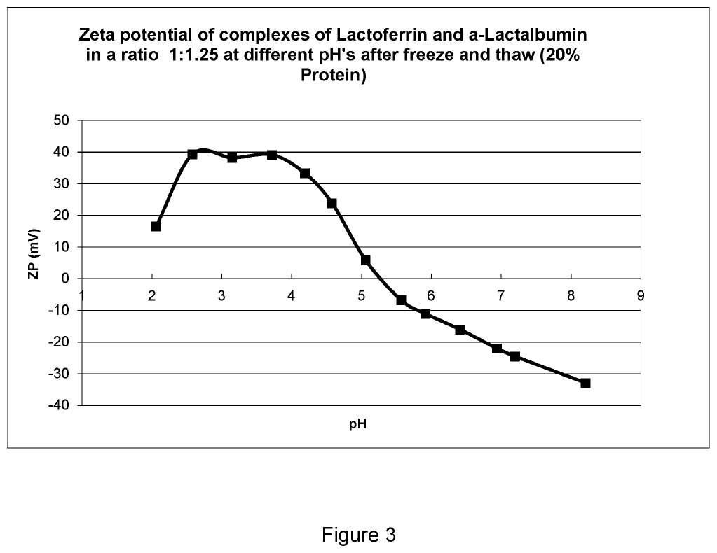

The variation of the Zeta-potential of complexes of LF and ALAC prepared at a 20% protein content in a 1:1.25 ratio at different pH's, after freezing and thawing is shown on FIG. 3. For Zeta-potential measurement, the sample was diluted to a 0.1% protein content. The samples were frozen overnight at −20° C. in 50 ml test tubes and left at ambient temperature for thawing.

The results show that the same Zeta-potential values are found though the complexes have undergone freezing and thawing, a physical process which is known to strongly affect protein structure.

In the present case, the complexes did not suffer degradation and showed a high stability. They are negatively charged in the range of pH 5.5 to 8.0.

### EXAMPLE 3

### Complexes of Lactoferrin (LF) and Beta-Lactoglobulin (Blg)

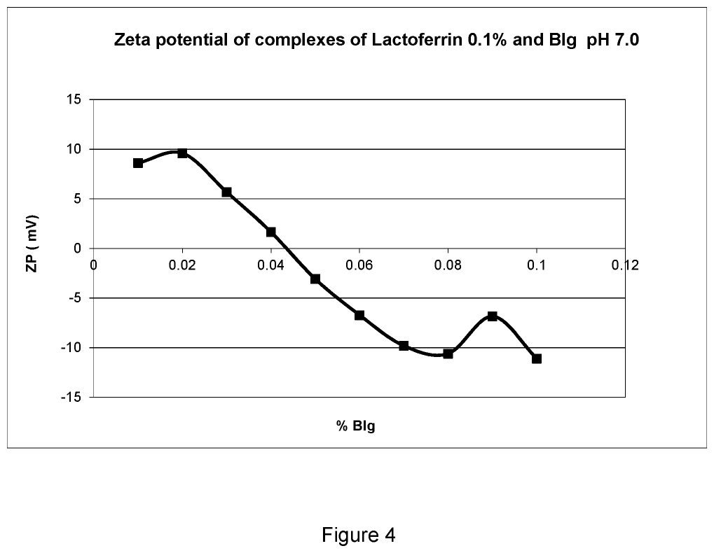

The variation of the Zeta-potential of complexes of LF (0.1%) and beta-lactoglobulin (Blg) for variable concentrations of Blg at pH 7.0 is shown on FIG. 4. Under the tested conditions, the complexes show a negative charge at ratios of LF:Blg ranging from 1:0.5 to 1:1.

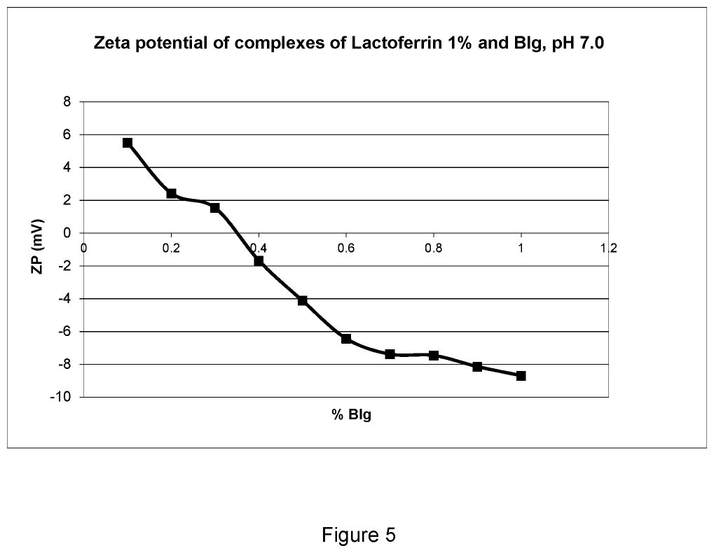

The variation of the Zeta-potential of complexes of LF (1%) and beta-lactoglobulin (Blg) for variable concentrations of Blg at pH 7.0 are shown on FIG. 5. Under the tested conditions, the complexes show a negative charge at ratios of LF:Blg ranging from 1:0.4 to 1:1.

### EXAMPLE 4

### Complexes of LF (0.1%) and Whey Protein Hydrolyzate (Lactry)

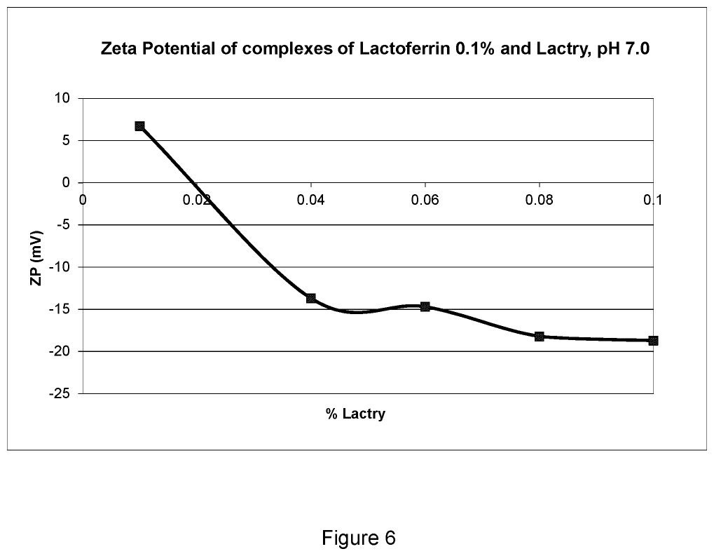

The variation of the Zeta-potential of complexes of LF (0.1%) and tryptic whey protein hydrolyzate for variable concentrations of Lactry at pH 7.0 is shown on FIG. 6. Under the tested conditions, the complexes show a negative charge at ratios of LF: whey protein hydrolyzate ranging from 1:0.4 to 1:21.

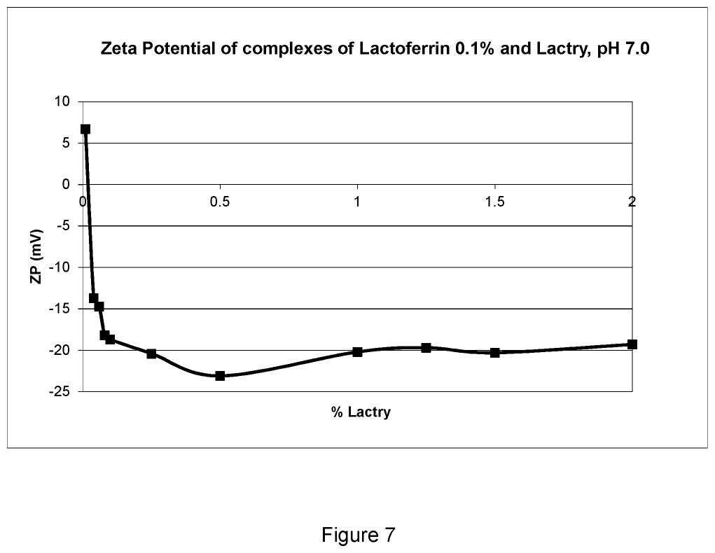

The variation of the Zeta-potential of complexes of LF (1%) and tryptic whey protein hydrolyzate for variable concentrations of Lactry at pH 7.0 is shown on FIG. 7. Under the tested conditions, the complexes show a negative charge at ratios of LF: whey protein hydrolyzate ranging from 1:0.5 to 1:1.

### EXAMPLE 5

### Complexes of Lactoferrin (LF) and Beta-Lactoglobulin (Blg) with Concentrations of LF of 2% and 10%

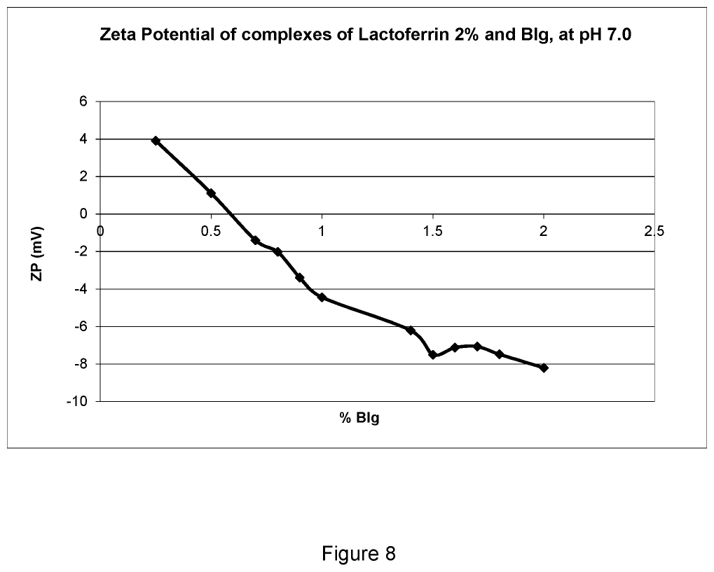

The variation of the Zeta-potential of complexes of LF (2%) and beta-lactoglobulin (Blg) for variable concentrations of Blg at pH 7.0 is shown on FIG. 8. Under the tested conditions, the complexes show a negative charge at ratios of LF: Blg ranging from 2:0.7 to 2:2.

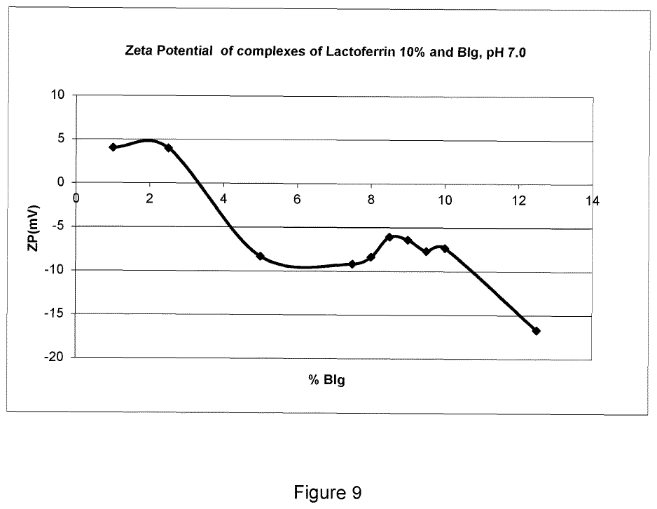

The variation of the Zeta-potential of complexes of LF (10%) and beta-lactoglobulin (Blg) for variable concentrations of Blg at pH 7.0 is shown on FIG. 9. Under the tested conditions, the complexes show a negative charge at ratios of LF: Blg ranging from 1:0.4 to 1:0.2.

### EXAMPLE 6

### Starter Infant Formula

An example of the composition of a nutritional formula (starter infant formula) according the invention is given in table 1 below.

TABLE 1

| | | per | per | per |
|---|---|---|---|---|
| Nutrients | Unit | 100 g | 100 kcal | liter |
| ENERGY | Kcal | 498 | 100 | 662 |
| ENERGY | Kj | 2081 | 418 | 2768 |
| WATER | g | 2.5 | 0.5 | 903 |
| ASH/MINERALS (total) | g | 3.9 | 0.7 | 5.2 |
| PROTEIN | g | 10.0 | 2.0 | 13.4 |
| Whey protein | g | 6.9 | 1.4 | 9.2 |
| Alpha lactalbumin | g | 1.7 | 0.3 | 2.3 |
| Lactoferrin | mg | 454 | 91.3 | 600 |
| Casein | g | 3.1 | 0.6 | 4.2 |
| FAT | g | 27.0 | 5.4 | 36.0 |
| 1-3-dioleyl-2-palmitoyl- | g | 4.0 | 0.8 | 5.3 |
| triglyceride | | | | |
| Linoleic acid | g | 3.9 | 0.7 | 5.2 |
| alpha-Linolenic acid | mg | 316 | 63.4 | 420 |
| Linoleic/alpha-Linolenic | | 12 | 12 | |
| acid ratio | | | | |
| Arachidonic acid (ARA) | mg | 94.7 | 19.0 | 126 |
| Docosahexaenoic acid | mg | 90.0 | 18.0 | 120 |
| (DHA) | | | | |
| ARA/DHA ratio | | 1.05 | 1.05 | |
| CARBOHYDRATES | | | | |
| Total carbohydrates | g | 56.3 | 11.3 | 75.0 |
| Available carbohydrates | g | 50.5 | 10.1 | 67.1 |
| Of which sugars | g | 50.5 | 10.1 | 67.1 |
| Of which lactose | g | 50.5 | 10.1 | 67.1 |
| OLIGOSACCHARIDES | g | 5.8 | 1.1 | 7.8 |
| NUCLEOTIDES (total) | mg | 19.5 | 3.9 | 26.0 |
| (mg) | | | | |
| CMP | mg | 9.7 | 2.00 | 13.0 |
| UMP | mg | 3.7 | 0.76 | 5.0 |
| AMP | mg | 3.0 | 0.60 | 4.0 |
| GMP | mg | 1.5 | 0.30 | 2.0 |
| IMP | mg | 1.5 | 0.30 | 2.0 |
| VITAMINS | | | | |
| Vitamin A | μg RE | 545 | 109 | 725 |
| Beta-Carotene | μg | 113 | 22.6 | 150 |
| Vitamin D | μg | 7.2 | 1.4 | 9.6 |
| Vitamin E | mg TE | 6.5 | 1.3 | 8.7 |
| Vitamin K1 | μg | 40.3 | 8.1 | 53.6 |
| Vitamin B1 | mg | 0.94 | 0.1 | 1.25 |
| Vitamin B2 | mg | 0.84 | 0.1 | 1.11 |
| Vitamin B6 | mg | 0.56 | 0.1 | 0.75 |
| Vitamin B12 | μg | 1.73 | 0.3 | 2.30 |
| Niacin | mg | 4.70 | 0.9 | 6.25 |
| Folic acid | μg | 75.1 | 15.1 | 100 |
| Pantothenic acid | mg | 2.82 | 0.5 | 3.75 |
| Biotin | μg | 18.8 | 3.7 | 25.0 |
| Vitamin C | mg | 67.6 | 13.5 | 90.0 |
| MINERALS | | | 0.37 | 2.5 |
| Ca | mg | 322 | 64.6 | 428 |
| P | mg | 180 | 36.2 | 240 |
| Ca/P ratio | | 1.7 | 1.7 | |
| Mg | mg | 55 | 11.0 | 72.6 |
| Fe | mg | 6.01 | 1.21 | 8.0 |
| Zn | mg | 5.4 | 1.08 | 7.13 |
| Cu | mg | 0.35 | 0.07 | 0.46 |
| Zn/Cu ratio | | 12.9 | 12.9 | |
| Mn | μg | 37.6 | 7.5 | 50.0 |
| I | μg | 75.2 | 15.1 | 100 |
| Na | mg | 131 | 26.2 | 174 |
| K | mg | 400 | 80.3 | 528 |
| Cl | mg | 326 | 65.4 | 433 |
| Se | μg | 15.0 | 3.03 | 20.0 |
| AMINOACIDS | | | | |
| L-Tyrosine | mg | 303 | 60.8 | 403 |
| L-Tryptophan | mg | 167 | 33.4 | 222 |
| OTHER SUBSTANCES | | | | |
| Taurine | mg | 35.3 | 7.1 | 47.0 |
| L-Carnitine | mg | 7.52 | 1.5 | 10.0 |
| Choline | mg | 123 | 24.7 | 164 |
| Inositol | mg | 33.8 | 6.7 | 45.0 |
| Lutein | μg | 87.0 | 17.0 | 116 |

### EXAMPLE 7

### Follow-Up Formula

An example of the composition of a nutritional formula (follow-up formula) according the invention is given in table 2 below.

TABLE 2

| | | per | per | per |
|---|---|---|---|---|
| Nutrients | Unit | 100 g | 100 kcal | liter |
| ENERGY | Kcal | 465 | 100 | 674 |
| ENERGY | Kj | 1949 | 419 | 2826 |
| WATER | g | 2.5 | 0.5 | 898.6 |
| ASH/MINERALS (total) | g | 4.7 | 1.0 | 6.8 |
| PROTEIN | g | 15 | 3.1 | 21.3 |
| Whey protein | g | 6.5 | 1.3 | 9.2 |
| Alpha lactalbumin | g | 0.9 | 0.2 | 1.3 |
| Lactoferrin | mg | 425 | 91.5 | 600 |
| Casein | g | 8.5 | 1.8 | 12.1 |
| FAT | g | 20.7 | 4.4 | 30.0 |
| 1-3-dioleyl-2-palmitoyl- | g | 4.0 | 0.9 | 5.8 |
| triglyceride | | | | |
| Linoleic acid | g | 3.0 | 0.6 | 4.3 |
| alpha-Linolenic acid | g | 0.29 | 0.06 | 0.4 |
| Linoleic/alpha-Linolenic | | 10.1 | 10.1 | |
| acid ratio | | | | |
| Arachidonic acid (ARA) | mg | 79.0 | 17.0 | 114.5 |
| Docosahexaenoic acid | mg | 72.5 | 15.6 | 105 |
| (DHA) | | | | |
| ARA/DHA ratio | | 1.09 | 1.09 | |
| CARBOHYDRATES | | | | |
| Total carbohydrates | g | 57.3 | 12.3 | 83.0 |
| Available carbohydrates | g | 52.3 | 11.2 | 75.8 |
| Of which lactose | g | 52.3 | 11.2 | 75.8 |
| Total fibers | g | 4.9 | 1.06 | 7.2 |
| OLIGOSACCHARIDES | g | 4.9 | 1.1 | 7.8 |
| NUCLEOTIDES (total) | mg | 17.9 | 3.8 | 26.0 |
| CMP | mg | 9.9 | 1.9 | 13.0 |
| UMP | mg | 3.4 | 0.7 | 5.0 |
| AMP | mg | 2.7 | 0.6 | 4.0 |
| GMP | mg | 1.4 | 0.30 | 2.0 |
| IMP | mg | 1.4 | 0.30 | 2.0 |
| VITAMINS | | | | |
| Vitamin A | μg RE | 407 | 87.6 | 590 |
| Beta-Carotene | μg Car | 103 | 22.6 | 150 |
| Vitamin D | μg | 7.7 | 1.6 | 11.2 |
| Vitamin E | mg TE | 4.6 | 0.99 | 6.7 |
| Vitamin K1 | μg | 30.3 | 6.5 | 44.0 |
| Vitamin B1 | mg | 0.43 | 0.09 | 1.25 |
| Vitamin B2 | mg | 1.2 | 0.26 | 1.11 |
| Vitamin B6 | mg | 0.34 | 0.07 | 0.75 |
| Vitamin B12 | μg | 1.24 | 0.26 | 2.30 |
| Niacin | mg | 4.7 | 1.01 | 6.63 |
| Folic acid | μg | 92.3 | 19.9 | 100 |
| Pantothenic acid | mg | 2.41 | 0.5 | 3.75 |
| Biotin | μg | 10.3 | 2.2 | 25.0 |
| Vitamin C | mg | 62.0 | 13.3 | 90.0 |
| MINERALS | | | | |
| Ca | mg | 523 | 113 | 759 |
| P | mg | 352 | 75.7 | 510 |
| Ca/P ratio | | 1.48 | 1.5 | |
| Mg | mg | 75 | 16.1 | 106 |
| Fe | mg | 8.3 | 1.78 | 12.0 |
| Zn | mg | 5.0 | 1.07 | 7.05 |
| Cu | mg | 0.20 | 0.04 | 0.29 |
| Zn/Cu ratio | | 20.4 | 20.4 | |
| Mn | μg | 241 | 51.8 | 349 |
| I | μg | 68.9 | 18.8 | 100 |
| Na | mg | 231 | 49.7 | 335 |
| K | mg | 450 | 96.8 | 634 |
| Cl | mg | 480 | 103.3 | 696 |
| Se | μg | 13.7 | 2.9 | 20.0 |
| OTHER SUBSTANCES | | | | |
| Taurine | mg | 32.4 | 6.9 | 47.0 |
| Choline | mg | 140.0 | 30.1 | 203 |
| Inositol | mg | 386 | 8.3 | 55.9 |
| Lutein | μg | 160 | 34.4 | 226 |

### EXAMPLE 8

### Infant Formula having an Energy Density less than 65 kcal/100 ml

A nutritional formula (starter infant formula) according the invention was prepared according to table 1 of example 6, except that the fat content was adjusted to obtain an energy density of 620 kcal per Liter.

1. A nutritional composition for infants or young children comprising proteins, carbohydrates and lipids, wherein said proteins comprise ionic complexes of lactoferrin with acid milk proteins, said complexes having a negative charge at the pH of the infant formula.

2. The composition of claim 1, wherein said protein is a mixture of whey protein and casein, wherein the ratio of whey protein to casein is between 50:50 and 80:20.

3. The composition of claim 1, wherein said acid milk protein is selected from the group consisting of alpha-lactalbumin (ALAC), beta-lactoglobulin (BLG), whey protein isolate (WPI), whey protein concentrate (WPC), whey protein hydrolysate, casein and combinations thereof.

4. The composition of claim 2, wherein whey protein is enriched with alpha-lactalbumin (ALAC).

5. The composition of claim 3, wherein the ALAC content in whey protein is at least 10% by weight.

6. The composition of claim 1, wherein lactoferrin in the form of ionic complexes of lactoferrin with acid milk proteins represent 1 to 20% of the whey protein.

7. The composition of claim 3, wherein in said ionic complex:
the weight ratio of lactoferrin to ALAC ranges from 1:0.25 to 1:3; and/or
the weight ratio of lactoferrin to BLG ranges from 1:0.25 to 1:3; and/or
the weight ratio of lactoferrin to casein ranges from 1:0.25 to 1:3.

8. The composition of claim 1, wherein the Zeta-potential of said complexes at pH 7.0 is from −3 mV to −20 mV measured for a total protein content of 0.1%.

9. The composition of claim 1, wherein the lipids are selected from the group consisting of linoleic acid, linolenic acid, arachidonic acid (ARA) and docosahexaenoic acid (DHA).

10. The composition of claim 1, wherein said carbohydrate consists of at least 90%.

11. The composition of claim 1, wherein, the amounts of Fe, Zn, Mg vitamin D, K or vitamin B12, are as follows:
Fe: 1 to 2 mg/100 kcal,
Zn: 0.7 to 1.5 mg/100 kcal,
Mg: 5.5 to 16.5 mg/100 kcal,
Vitamin D: 1 to 2.6 μg/100 kcal,
K: 70 to 170 mg/100 kcal, and
vitamin B12: 0.25 to 1.5 μg/100 kcal.

12. The composition of claim 1, wherein the energy density of the composition is less than 65 kcal/100 ml.

13. A method for use in providing a satiety feeling and/or a better sleep and/or limiting nocturnal awaking in infants or young children comprising the step of administering a nutritional composition for infants or young children comprising proteins, carbohydrates and lipids, wherein said proteins comprise ionic complexes of lactoferrin with acid milk proteins, said complexes having a negative charge at the pH of the infant formula to an infant or young child in need of same.

14. The method according to claim 13, wherein said composition provides slow digestion of proteins.

15. The method according to claim 12 in infants or young children having sleep disorders with respect to sleep quality or sleep time, for improvement of sleep quality or pattern.

16. The method according to claim 12, wherein the improvement of sleep quality or pattern comprises the reduction of the number of episodes of wake states and/or the reduction of sleep fragmentation and/or longer nights without being unwillingly awake and/or a more peaceful sleep and/or better ability to fall asleep.

17. The method according to claim 12, wherein said composition is starter infant formula or a follow-up infant formula.
